(19) United States
(12) Patent Application Publication
(10) Pub. No.: US 2026/0345193 A1
(43) Pub. Date: Nov. 5, 2026

(54) A ROTATIONAL ATHERECTOMY DEVICE

(71) Applicant: CLEARSTREAM TECHNOLOGIES LIMITED, Enniscorthy, County Wexford (IE)

(72) Inventors: Allan RONAN, Enniscorthy, County Wexford (IE); Eoin CONNAUGHTON, Gorey (IE); Artur SPODOBALSKI, Blackwater (IE)

(21) Appl. No.: 19/121,227

(22) PCT Filed: Oct. 18, 2022
(86) PCT No.: PCT/EP2022/078932

§ 371 Date: Apr. 15, 2025

(51) Int. Cl. A61B 17/3207; A61B 17/00; A61B 17/32

(52) U.S. Cl. CPC A61B 17/320725; A61B 2017/00398; A61B 2017/320004

(57) ABSTRACT

A rotational atherectomy device for cutting or abrading tissue. The atherectomy device comprises a flexible rotatable drive shaft. The atherectomy device further comprises an abrasive element disposed on a non-precurved section of the flexible rotatable drive shaft. The atherectomy device further comprises a control wire fixed to a distal end of the flexible rotatable drive shaft. Proximal movement of the control wire relative to the flexible rotatable drive shaft moves the abrasive element from a radially contracted configuration to a radially extended configuration.

### TECHNICAL FIELD

The present disclosure relates to a rotational atherectomy device for cutting or abrading tissue; and a method of cutting or abrading tissue using an atherectomy device.

### BACKGROUND

Atherosclerosis is a condition where calcified plaque deposits in the walls of a blood vessel lead to narrowing of the blood vessel and increased blood pressure. In severe cases, this can result in peripheral artery disease, coronary artery disease, tissue death or stroke.

Atherosclerosis can be treated in a number of ways such as by angioplasty, where a balloon is used to force the expansion of the blood vessel to allow improved blood flow, a vascular bypass, where a surgical procedure is performed to redirect the blood flow to circumvent the diseased area of the blood vessel, or atherectomy.

Atherectomy is a non-surgical procedure to open blocked blood vessels using a device at the distal end of a catheter to cut or grind atherosclerotic plaque from the walls of the blood vessel.

A rotational atherectomy device uses a rotating abrasive head to cut or grind the atherosclerotic plaque from the walls of the blood vessel. A number of existing rotational atherectomy devices incorporate an abrasive head of fixed diameter. In order to treat larger diameter vessels, the abrasive head is offset towards one side of the vessel wall and the lesion is treated with multiple axial passes. This makes the treatment procedure longer and more complex. Another type of expandable rotational atherectomy device is disclosed, for example, in US 2010/0121361 A1 where an abrasive section is disposed on a pre-curved section of a flexible drive shaft which expands when the pre-curved section is moved distally out of a catheter. However, this type of expanding atherectomy device does not allow a vessel with a diameter smaller than the radial extent of the pre-curved section to be treated. This type of atherectomy device also does not allow a physician to easily adjust the amount of extension of the abrasive head.

There is hence a need in the art for a new type of rotational atherectomy device which allows controlled and precise radial extension of the abrasive head and therefore reduce the overall treatment time as larger vessels can be treated in fewer axial passes of the abrasive head.

There is also a need in the art for a new type of rotational atherectomy device which can accurately treat vessels with a small diameter.

### SUMMARY

In a first aspect of the present disclosure, there is provided a rotational atherectomy device for cutting or abrading tissue. The atherectomy device comprises a flexible rotatable drive shaft. The atherectomy device further comprises an abrasive element disposed on a non-precurved section of the flexible rotatable drive shaft. The atherectomy device further comprises a control wire fixed to a distal end of the flexible rotatable drive shaft. Proximal movement of the control wire relative to the flexible rotatable drive shaft moves the abrasive element from radially contracted configuration to a radially extended configuration.

In some embodiments, this may allow controlled and precise radial extension of the abrasive element and therefore reduce the overall treatment time as larger vessels can be treated in fewer axial passes of the abrasive head.

In some embodiments, this may further allow the atherectomy device to accurately treat vessels with a small diameter.

Throughout this disclosure, the term “non-precurved section” will be used to refer a section which is straight in a relaxed state when no forces are applied to the section.

Throughout this disclosure, the term “radially extended configuration” will be used to refer to a configuration in which the abrasive element extends radially further from a central longitudinal axis of the device than in the “radially contracted configuration”.

The control wire and flexible rotatable drive shaft may be configured to rotate together.

The rotational atherectomy device may further comprise a coupling element positioned proximally of the abrasive element which couples the control wire to the flexible rotatable drive shaft.

In some embodiments, this may allow more accurate control over the radial extension of the abrasive element.

The coupling element may allow longitudinal movement of the control wire relative to the flexible rotatable drive shaft.

In some embodiments, this may allow more accurate control over the radial extension of the abrasive element.

A distal section of the control wire may be stiffer than the flexible rotatable drive shaft. The distal section may be between the coupling element and the distal end.

In some embodiments, this may allow more accurate control over the radial extension of the abrasive element.

The control wire may be a flexible control wire.

In some embodiments, this may allow the rotational atherectomy device to more easily navigate tortuous blood vessel anatomy.

The abrasive element may comprise one or more abrasive surfaces.

In some embodiments, this may result in more effective abrading of plaque from a vessel wall.

The abrasive element may have a substantially spherical shape.

In some embodiments, this may allow for accurate and targeted treatment of a small section of a vessel wall.

The abrasive element may have a substantially ellipsoid shape.

In some embodiments, this may allow a longer section of a vessel wall to be treated.

The abrasive element may have a substantially cylindrical shape.

In some embodiments, this may allow a longer section of a vessel wall to be treated.

The rotational atherectomy device may further comprise an outer shaft which may be disposed over the flexible rotatable drive shaft and the control wire.

In some embodiments, this may minimise unwanted contact, such as by abrasive elements with a vessel wall, during introduction of the rotational atherectomy device into a vessel and advancement to a treatment site.

The outer shaft may be a flexible outer shaft.

In some embodiments, this may allow the rotational atherectomy device to more easily navigate tortuous blood vessel anatomy.

The outer shaft may be moveable relative to the control wire and the flexible rotatable drive shaft.

In some embodiments, this may allow the abrasive head to be protected by the outer shaft during introduction of the rotational atherectomy device into a vessel and advancement to a treatment site.

The rotational atherectomy device may further comprise a control unit connected to a proximal end of the flexible rotatable drive shaft.

The control unit may comprise a motor for providing rotation to the flexible rotatable drive shaft.

In some embodiments, this may result in more effective abrading of plaque.

The control unit may comprise a rotation control mechanism for controlling the speed of rotation of the motor.

In some embodiments, this may result in more effective control over the abrasion process.

The control unit may be connected to the control wire and may comprise an extension control mechanism for moving the control wire relative to the flexible rotatable drive shaft and controlling the amount of radial extension of the abrasive element.

In some embodiments, this may allow for precise control over the radial extension of the abrasive element.

The extension control mechanism may comprise an adjustment screw connected to the control wire.

In some embodiments, this may allow for more precise control over the radial extension of the abrasive element.

In a second aspect of the present disclosure, there is provided a method of cutting or abrading tissue using a rotational atherectomy device, the rotational atherectomy device comprising a flexible rotatable drive shaft, an abrasive element disposed on a non-precurved section of the flexible rotatable drive shaft, and a control wire fixed to a distal end of the flexible rotatable drive shaft. The method comprises introducing the rotational atherectomy device into a blood vessel; advancing the rotational atherectomy device to a position in the blood vessel where tissue is to be abraded; moving the control wire relative to the rotatable flexible drive shaft to move the abrasive element from a radially contracted configuration to a radially extended configuration; and rotating the abrasive element to cut or abrade the tissue.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a rotational atherectomy device having an abrasive element according to the present disclosure;

FIG. 2A shows a distal end of the rotational atherectomy device of FIG. 1 with the abrasive element in a radially contracted configuration;

FIG. 2B shows a distal end of the rotational atherectomy device of FIG. 1 with the abrasive element in a radially extended configuration;

FIG. 3A shows a distal end of the rotational atherectomy device of FIG. 1 disposed in an artery with the abrasive element in a radially contracted configuration;

FIG. 3B shows the distal end of the rotational atherectomy device of FIG. 1 disposed in an artery with the abrasive element in a radially extended configuration;

FIG. 3C shows a distal end of the rotational atherectomy device of FIG. 1 disposed in an artery with the abrasive element in a radially extended configuration and rotating. s

FIG. 4A shows an embodiment of an abrasive element for a rotational atherectomy device having an ellipsoidal shape;

FIG. 4B shows an embodiment of an abrasive element for a rotational atherectomy device having a spherical shape; and

FIG. 4C shows an embodiment of an abrasive element for a rotational atherectomy device having a cylindrical shape.

### DETAILED DESCRIPTION

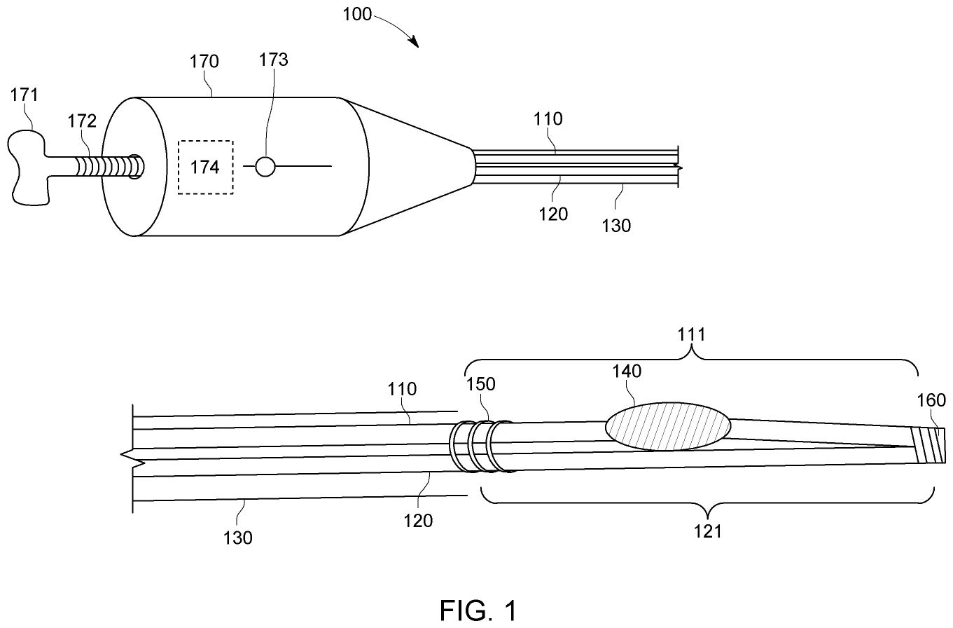

FIG. 1 shows a rotational atherectomy device 100 for cutting or abrading tissue, specifically atherosclerotic plaque in a blood vessel.

The rotational atherectomy device comprises a drive shaft 110, a control wire 120 and an abrasive element 140. The drive shaft 110 has a non-precurved section 111 which may be positioned near a distal end of the drive shaft 110. The non-precurved section 110 is straight when no forces are applied to it, however, it may bend or curve when a force is applied to it. The abrasive element 140 is positioned on the non-precurved section 111 of the drive shaft 110. The drive shaft 110, including the abrasive element 140, is rotatable to allow the abrasive element 140 to cut or abrade atherosclerotic plaque from a vessel wall. Furthermore, the drive shaft 110 is flexible to allow radial extension of the abrasive element 140 and to allow it to navigate tortuous blood vessel anatomy when advancing the rotational atherectomy device 100 though a vessel to a treatment site.

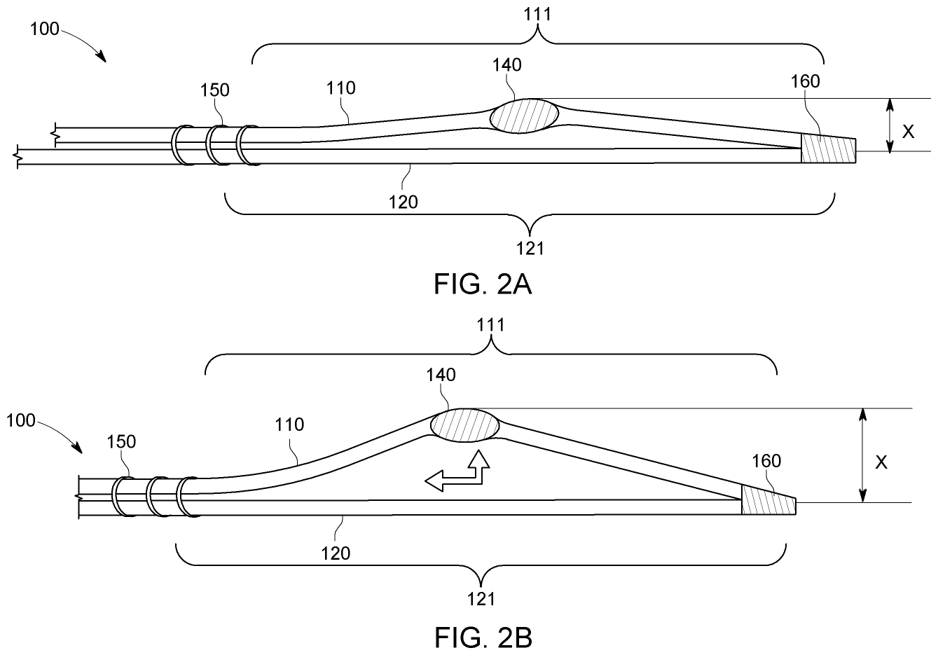

A distal end of the control wire 120 is fixed to a distal end of the drive shaft 110 with a fixing element 160, which may be an adhesive or a mechanical crimp, for example. The control wire 120 may be configured to rotate together with the drive shaft 120. The abrasive element 140 can be moved between a radially contracted configuration, as shown in FIG. 1 (see also FIG. 2A), and a radially extended configuration (see FIG. 2B) by moving the control wire 120 longitudinally relative to the drive shaft 110. The amount of radial extension of the abrasive element 140 can be controlled by controlling the longitudinal position of the control wire 120 relative to the drive shaft 110. The abrasive element 140 may comprise one or more abrasive surfaces. For example, the entire abrasive element 140 may be covered with one abrasive surface, such as an abrasive coating. The abrasive surface(s) may be rough to allow them to scrape or abrade plaque from the vessel wall when the drive shaft is rotating.

The abrasive element 140 may be made from a number of suitable materials such as, but not limited to, a cobalt base alloy, oxidised zirconium, or tungsten. The control wire 120 may be made from a suitable metallic or polymeric material having high tensile strength and low longitudinal stretch to provide predictable operation behaviour, for example, stainless steel or nitinol. The drive shaft 110 may be formed, for example, as a nitinol tube, a stainless steel coil, or other composite layered polymer or metal material.

The atherectomy device 100 may further comprise a coupling element 150 positioned proximal of the abrasive element 140 and which couples the drive shaft 110 with the control wire 120. The coupling element 150 may be in the form of one or more coupling rings or a coupling sleeve, for example, which hold the drive shaft 110 and control wire 120 together. The coupling element 150 prevents transverse movement of the drive shaft 110 and the control wire 120 relative to each other but allows the control wire 120 to move longitudinally with respect to drive shaft 110. The control wire 120 may be flexible to allow it to more easily navigate through blood vessel anatomy. However, a distal section 121 of the control wire 120, between the coupling element 150 and the fixing element 160, may be stiff, and in particular, stiffer than the non-precurved section 111 of the drive shaft 110. This distal section 121 of the control wire 120 may not bend easily, or not as easily as the non-precurved section 111. When the control wire 120 is pulled proximally relative to the drive shaft 110, the stiff distal section 121 of the control wire 120 will not bend and rather the non-precurved section 111 of the flexible drive shaft 110 will bend radially outwards, causing the abrasive element 140 to extend radially.

The rotational atherectomy device 100 may have an outer shaft 130 disposed over the drive shaft 110 and the control wire 120. The outer shaft 130 may prevent transverse movement of the drive shaft 110 and the control wire 120 relative to each other. Furthermore, the outer shaft 130 may be longitudinally moveable with respect to the drive shaft 110 and control wire 120. For example, the outer shaft 130 may be moved distally to cover the abrasive element 140 during introduction into and advancement of the rotational atherectomy device 100 in a blood vessel to protect the abrasive head 140 and minimise damage to the vessel wall. At the treatment site, the outer shaft 130 may then be moved proximally to uncover the abrasive element 140 and allow the abrasive element 140 to cut or abrade plaque from the vessel wall. The outer shaft 130 may be configured to rotate together with the drive shaft 110 and the control wire 120.

The rotational atherectomy device 100 may further comprise a control unit 170 disposed at a proximal end of the flexible rotatable drive shaft 110 and control wire 110. The control unit 170 may be in the form of a handle and may comprise a motor 174 connected to the drive shaft 110 and the control wire 110 for providing rotation to the drive shaft 110 and control wire 120. The control handle 170 may comprise a rotation control mechanism for controlling the speed of the rotation of the motor 174 and therefore the speed of rotation of the abrasive element 140. The rotation control mechanism 173 may be in the form of a linear slider 173. The control unit 170 may further comprise an extension control mechanism which is connected to the control wire 120. As shown in FIG. 1, the extension control mechanism may be in the form of an adjustment screw 171 comprising a thread 172. By rotating the adjustment screw 171, the control wire 120 may be moved longitudinally relative to the drive shaft 110 to control the amount of radial extension of the abrasive element 140. The thread 172 on the adjustment screw 171 may allow for very precise control over the amount of movement of the control wire 110 and therefore may allow precise control over the radial extension of the abrasive element 140.

FIG. 2A shows a distal end of the rotatable atherectomy device 100 where the abrasive element 140 is in the radially contracted configuration. In the radially contracted configuration, a radial extent x of the abrasive element 140 (i.e. the radial distance between the abrasive element 140 and a longitudinal central axis of the device 100) is small and the non-precurved section 111 is substantially straight. In the radially contracted configuration, the rotational atherectomy device 100 can more easily be advanced through a blood vessel and manoeuvre tortuous blood vessel anatomy. The small radial extent x in the radially contracted configuration also allows a vessel with a very small diameter to be treated.

FIG. 2B shows the rotatable atherectomy device where the abrasive element 140 is in the radially extended configuration. In the radially extended configuration, the radial extent x of the abrasive element 140 is greater than in the radially contracted configuration. The non-precurved section 111 is curved radially outwards away from the control wire 120. The greater radial extent x of the abrasive element allows a vessel with a greater diameter to be treated.

In order to move the abrasive element 140 from the radially contracted configuration to the radially extended configuration, the control wire 120 is pulled proximally relative to the drive shaft 110. This may be done, for example, by turning the adjustment screw 171. Due to the stiffer distal section 121 of the control wire 120, proximal movement of the control wire 120 results in the more flexible non-precurved section 111 curving radially outward which pushes the abrasive element 140 radially outward. The radial extent x of the abrasive element 140 may be precisely controlled by controlling the amount of proximal movement of the control wire 120 relative to the drive shaft 110 with the adjustment screw 171.

In order to move the abrasive element from the radially extended configuration back to the radially contracted configuration, the control wire 120 is moved distally relative to the drive shaft 110, for example, by turning the adjustment screw 171 in the opposite direction.

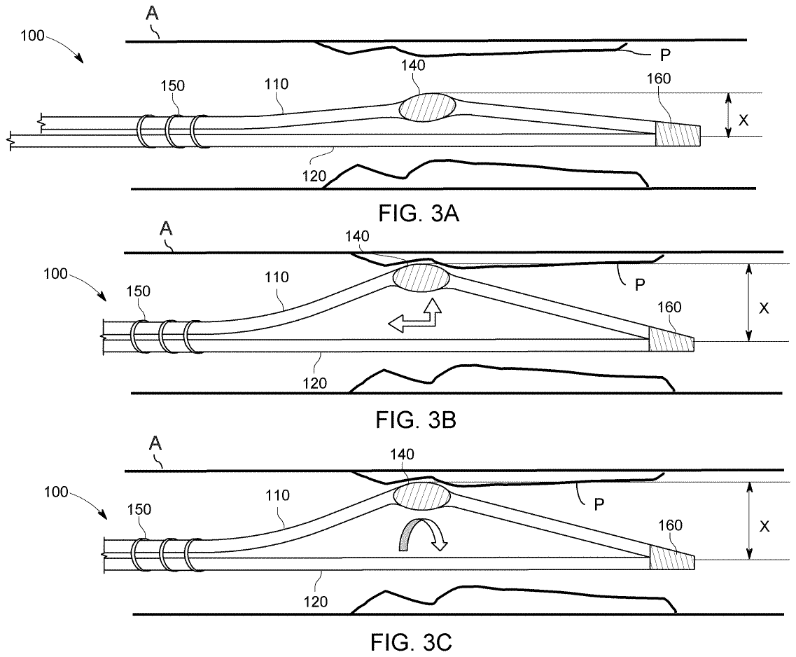

FIGS. 3A-C illustrate a method of using the rotational atherectomy device 100 in order to remove atherosclerotic plaque P from the walls of an artery A.

Firstly, the rotational atherectomy device 100 is introduced into the artery A through an access site and advanced to a treatment site where plaque P is to be removed from the artery walls. During the introduction and advancement, the abrasive element 140 may be in the radially contracted configuration and the outer shaft 130 may be in a distally advanced position so that it covers the abrasive element 140. Once the distal end of the rotational atherectomy device 100 is positioned at the treatment site, as shown in FIG. 3A, the outer shaft 130 may be pulled back to expose the abrasive element 140. A practitioner can then move the abrasive element 140 from the radially contracted configuration to the radially extended configuration, as shown in FIG. 3B, and adjust the radial extent x of the abrasive element 140 to match the diameter of the vessel to be treated, or other desired diameter, by moving the control wire 120 proximally, for example, via adjustment screw 171.

The practitioner may then turn on the motor 174 and can adjust the speed of rotation of the motor 174, for example, through the linear slider 173 of the rotation control mechanism. As shown in FIG. 3C, the motor 174 imparts rotation to the drive shaft 110 and the abrasive element 140. The rotating abrasive element 140 contacts the vessel wall removes the plaque P from the wall of the artery A.

Once the plaque P has been removed from the vessel wall, the practitioner can move the abrasive element 140 from the radially expanded configuration to the radially contracted configuration by pushing the control wire 120 distally, for example, via adjustment screw 171.

The outer sheath 130 may then be moved distally to cover the abrasive element 140 and the rotational atherectomy device 100 may be removed from the artery A.

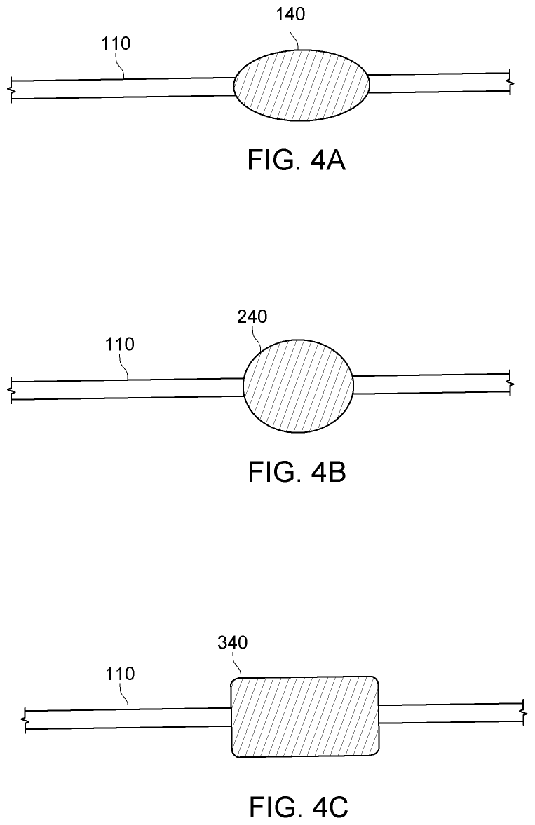

FIGS 4A-C illustrate different embodiments of the abrasive head.

FIG. 4A shows the abrasive head 140 having a substantially ellipsoid shape. The ellipsoid shape of the abrasive head 140 allows a greater contact area between the abrasive head 140 and the vessel wall whilst still allowing specific sections of the vessel to be precisely targeted. FIG. 4B shows an embodiment of an abrasive head 240 having a substantially spherical shape. The spherical shape of abrasive head 240 results in a smaller contact area of the abrasive head 240 with a vessel wall. This may allow the abrasive head 240 to more precisely target specific sections of a vessel wall. FIG. 4C shows an embodiment of an abrasive head 340 having a substantially cylindrical shape. The cylindrical shape of the abrasive head 340 results in a greater contact area of the abrasive head 340 with a vessel wall. This may allow the abrasive head 340 to treat a bigger section of the vessel wall and therefore speed up the procedure.

Each of the abrasive heads 140, 240, 340 may be used with the rotational atherectomy device 100.

Various modifications will be apparent to those skilled in the art.

The drive shaft 110 and the control wire 120 may not rotate together. The drive shaft 110 may be configured to rotate about the control wire 120.

The drive shaft 110 is not limited to any specific material.

The rotatable atherectomy device 100 may not comprise a coupling element 150. The transverse movement of the drive shaft 110 relative to the control wire 120 may be prevented, for example, by the outer shaft 130.

The control wire 120 may not be stiffer than the drive shaft 110. They may have a similar stiffness or the drive shaft 110 may even be stiffer than the control wire 120.

The control wire 120 is not limited to any specific material.

The abrasive element 140 is not limited to any specific shape and may be substantially spherical, spheroidal, cylindrical or any other suitable shape.

The abrasive element 140 is not limited to any specific material.

The rotational atherectomy device 100 may not comprise an outer shaft 130.

The outer shaft 130 may not be moveable relative to the control wire 120 and drive shaft 110.

The rotational atherectomy device 100 may not comprise a control unit 170.

The control unit 170 may not comprise a rotation control mechanism. The rotation control mechanism is not limited to a linear slider 173 but may comprise any suitable mechanism such a rotatable knob, for example.

The control unit 170 may not comprise an extension control mechanism 171.

The extension control mechanism 171 is not limited to an adjustment screw 171 but may comprise any other suitable mechanism, such as a linear slider, a rotating wheel, or a rotating knob, for example.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A rotational atherectomy device for cutting or abrading tissue, the atherectomy device comprising:
a flexible rotatable drive shaft;
an abrasive element disposed on a non-precurved section of the flexible rotatable drive shaft; and
a control wire fixed to a distal end of the flexible rotatable drive shaft,
wherein proximal movement of the control wire relative to the flexible rotatable drive shaft moves the abrasive element from a radially contracted configuration to a radially extended configuration.

2. The rotational atherectomy device of claim 1, wherein control wire and flexible rotatable drive shaft are configured to rotate together.

3. The rotational atherectomy device of claim 1, further comprising a coupling element positioned proximally of the abrasive element which couples the control wire to the flexible rotatable drive shaft.

4. The rotational atherectomy device of claim 3, wherein the coupling element allows longitudinal movement of the control wire relative to the flexible rotatable drive shaft.

5. The rotational atherectomy device of claim 3, wherein a distal section of the control wire, between the coupling element and the distal end, is stiffer than the flexible rotatable drive shaft.

6. The rotational atherectomy device of claim 1, wherein the control wire is a flexible control wire.

7. The rotational atherectomy device of claim 1, wherein the abrasive element comprises one or more abrasive surfaces.

8. The rotational atherectomy device of preceding claim 1, wherein the abrasive element has a substantially spherical shape.

9. The rotational atherectomy device of claim 1, wherein the abrasive element has a substantially ellipsoid shape.

10. The rotational atherectomy device of claim 1, wherein the abrasive element has a substantially cylindrical shape.

11. The rotational atherectomy device of claim 1, further comprising an outer shaft, which is disposed over the flexible rotatable drive shaft and the control wire.

12. The rotational atherectomy device of claim 11, wherein the outer shaft is a flexible outer shaft.

13. The rotational atherectomy device of claim 11, wherein the outer shaft is moveable relative to the control wire and the flexible rotatable drive shaft.

14. The rotational atherectomy device of claim 1, further comprising a control unit connected to a proximal end of the flexible rotatable drive shaft.

15. The rotational atherectomy device of claim 14, wherein the control unit comprises a motor for providing rotation to the flexible rotatable drive shaft.

16. The rotational atherectomy device of claim 14, wherein the control unit comprises a rotation control mechanism for controlling the speed of rotation of the motor.

17. The rotational atherectomy device of claim 14, wherein the control unit is connected to the control wire and comprises an extension control mechanism for moving the control wire relative to the flexible rotatable drive shaft and controlling the amount of radial extension of the abrasive element.

18. The rotational atherectomy device of claim 17, wherein the extension control mechanism comprises an adjustment screw connected to the control wire.

19. A method of cutting or abrading tissue using a rotational atherectomy device, the rotational atherectomy device comprising a flexible rotatable drive shaft, an abrasive element disposed on a non-precurved section of the flexible rotatable drive shaft, and a control wire fixed to a distal end of the flexible rotatable drive shaft; the method comprising:
introducing the rotational atherectomy device into a blood vessel;
advancing the rotational atherectomy device to a position in the blood vessel where tissue is to be abraded;
moving the control wire relative to the rotatable flexible drive shaft to move the abrasive element from a radially contracted configuration to a radially extended configuration;
rotating the abrasive element to cut or abrade the tissue.
